(19) United States
(12) Patent Application Publication
(10) Pub. No.: US 2006/0142858 A1
(43) Pub. Date: Jun. 29, 2006

(54) Expandable implants for spinal disc replacement

(76) Inventors: Dennis Colleran, North Attleboro, MA (US); Carolyn Rogers, Frisco, TX (US); Justin Dye, Mansfield, MA (US)

Correspondence Address: CARR LLP (IST), 670 FOUNDERS SQUARE, 900 JACKSON STREET, DALLAS, TX, 75202, US

(21) Appl. No.: 11/303,311

(22) Filed: Dec. 16, 2005

Related U.S. Application Data
(60) Provisional application No. 60/637,312, filed on Dec. 16, 2004.
(60) Provisional application No. 60/660,422, filed on Mar. 10, 2005.
(60) Provisional application No. 60/700,861, filed on Jul. 20, 2005.

(51) Int. Cl. A61F 2/44

(52) U.S. Cl. 623/17.11

(57) ABSTRACT

Multiple embodiments of the present invention provide methods and apparatuses for maintaining spacing between neighboring vertebrae, while minimizing the size of the surgical opening required. In one embodiment, an expandable spinal implant is made having movable parts that can arranged so as to have a small maximum cross-sectional width so that the cage can be inserted through a smaller surgical opening and then expanded to a full size assembly between the vertebrae.

### CROSS-REFERENCED APPLICATIONS

This application claims priority to co-pending, and commonly assigned U.S. provisional applications Ser. No. 60/637,312, entitled “MEDICAL IMPLANT, TOOLS, SYSTEM, METHOD, AND SURGICAL KIT,” filed Dec. 16, 2004; U.S. provisional application Ser. No. 60/660,422, entitled “MEDICAL IMPLANT SYSTEM AND METHOD OF USE,” filed Mar. 10, 2005, and to co-pending and commonly assigned U.S. provisional application Ser. No. 60/700,861, entitled “EXPANDABLE SPINAL INTERBODY CAGE,” filed Jul. 20, 2005, the disclosures of which are hereby incorporated

### BACKGROUND

1. Field of the Invention

This disclosure relates to systems and methods for treating diseases of of human spines, and, more particularly, to interbody implant devices.

2. Description

The inter-vertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress, and in doing so pressure is exerted on nerves extending from the spinal cord by this reduced inter-vertebral spacing. Various other types of nerve problems may be experienced in the spine, such as exiting nerve root compression in neural foramen, passing nerve root compression. A few medical procedures have been devised to alleviate such nerve compression and the pain that results from nerve pressure. Many of these procedures revolve around attempts to prevent the vertebrae from moving too close to each other by surgically removing an improperly functioning disc and replacing it with a lumber interbody fusion (“LIF””) device. Although prior interbody devices, including LIF cage devices, may be effective at improving patient condition, the vertebrae of the spine, body organs, the spinal cord, other nerves, and other adjacent bodily structures make obtaining surgical access to the location between the vertebrae where the LIF cage is to be installed difficult.

It would be desirable to reduce the size of the LIF cage to minimize the size for the required surgical opening for installation of the LIF cage, while maintaining high strength, durability and reliability of the LIF cage device.

### SUMMARY

Certain aspects of the present invention provide methods and apparatuses for maintaining spacing between neighboring vertebrae, while minimizing the size of the surgical opening required. In one aspect, an LIF cage is made having movable parts that can arranged so as to have a small maximum cross-sectional width so that the cage can be inserted through a smaller surgical opening and then expanded to a full size assembly between the vertebrae.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1A is a perspective view of the first and second parts of one embodiment of an interconnecting multi-part LIF cage having a curved interconnecting side;

FIG. 1B is a plan view of the first and second parts of the interconnecting multi-part LIF cage

FIG. 1C is a side view of the back portion of the second part of the interconnecting multi-part LIF cage;

FIG. 1D is a perspective view of the second part of the interconnecting multi-part LIF cage;

FIG. 1E is a perspective view of the first part of the interconnecting multi-part LIF cage;

FIG. 2A is a perspective view of the first and second parts of an alternative embodiment of an interconnecting multi-part LIF cage having a linear interconnecting side;

FIG. 2B is a plan view of the first and second parts of the interconnecting multi-part LIF cage

FIG. 2C is a side view of the back portion of the second part of the interconnecting multi-part LIF cage;

FIG. 2D is a perspective view of the second part of the interconnecting multi-part LIF cage;

FIGS. 2E is a perspective view of the first part of the interconnecting multi-part LIF cage;

FIG. 3 is a perspective view of the first and second parts, partially connected, of an interconnecting multi-part LIF cage having a linear interconnecting side;

FIG. 4 is a perspective view of the first and second parts, partially connected, of an interconnecting multi-part LIF cage having a curved interconnecting side;

FIG. 5 is a perspective view of one embodiment of an expandable cage, wherein the cage has multiple sliding parts;

FIG. 6 is a perspective view of one sliding part of the expandable cage;

FIG. 7 is a perspective view of a band which can restrain the expandable cage;

FIG. 8A is a perspective view of the expandable cage with a band placed around the circumference of the cage;

FIG. 8B is a plan view of the expandable cage;

FIG. 9A is a perspective view of an alternative embodiment of an expandable cage;

FIG. 9B is a plan view of the expandable cage;

FIG. 9C is a side view of the expandable cage;

FIG. 10A is a perspective view of an alternative embodiment of an expandable cage in an expanded state;

FIG. 10B is a plan view of the expandable cage in an expanded state;

FIG. 10C is a perspective view of the expandable cage in a contracted state;

FIG. 10D is a plan view of the expandable cage in a contracted state;

FIG. 10E is a side view of the expandable cage;

FIG. 11A is a perspective view of an alternative embodiment of an expandable cage in an expanded state;

FIG. 11B is a plan view of the expandable cage in an expanded state;

FIG. 11C is a perspective view of the expandable cage in a contracted state;

FIG. 11D is a plan view of the expandable cage in a contracted state;

FIG. 11E is a side view of the expandable cage;

FIG. 12A is a perspective view of one embodiment of an accordion-configuration expandable cage in its final configuration;

FIG. 12B is a plan view of the accordion-configuration expandable cage in its final configuration;

FIG. 12C is a side view of the accordion-configuration expandable cage in its final configuration;

FIG. 12D is a perspective view of the expandable cage, where the cage is partially folded towards its final configuration;

FIG. 12E is a plan view of the expandable cage, where multiple hinged parts are arranged longitudinally in a line;

FIG. 13A is a perspective view of an alternative embodiment of an accordion-configuration expandable cage in its final configuration;

FIG. 13B is a plan view of the accordion-configuration expandable cage in its final configuration;

FIG. 13C is a side view of the accordion-configuration expandable cage in its final configuration;

FIG. 13D is a perspective view of the expandable cage, where the cage is partially folded towards its final configuration;

FIG. 13E is a plan view of the expandable cage, where multiple hinged parts are arranged longitudinally in a line;

FIG. 14A is a perspective view of one embodiment of a spiral-configuration expandable cage in its final configuration;

FIG. 14B is a plan view of the spiral-configuration expandable cage in its final configuration; and

FIG. 14C is a perspective view of the expandable cage, where the cage is arranged longitudinally in a line.

### DETAILED DESCRIPTION

In the following discussion, numerous specific details are set forth to provide a thorough understanding of the present invention. However, those skilled in the art will appreciate that the present invention may be practiced without such specific details.

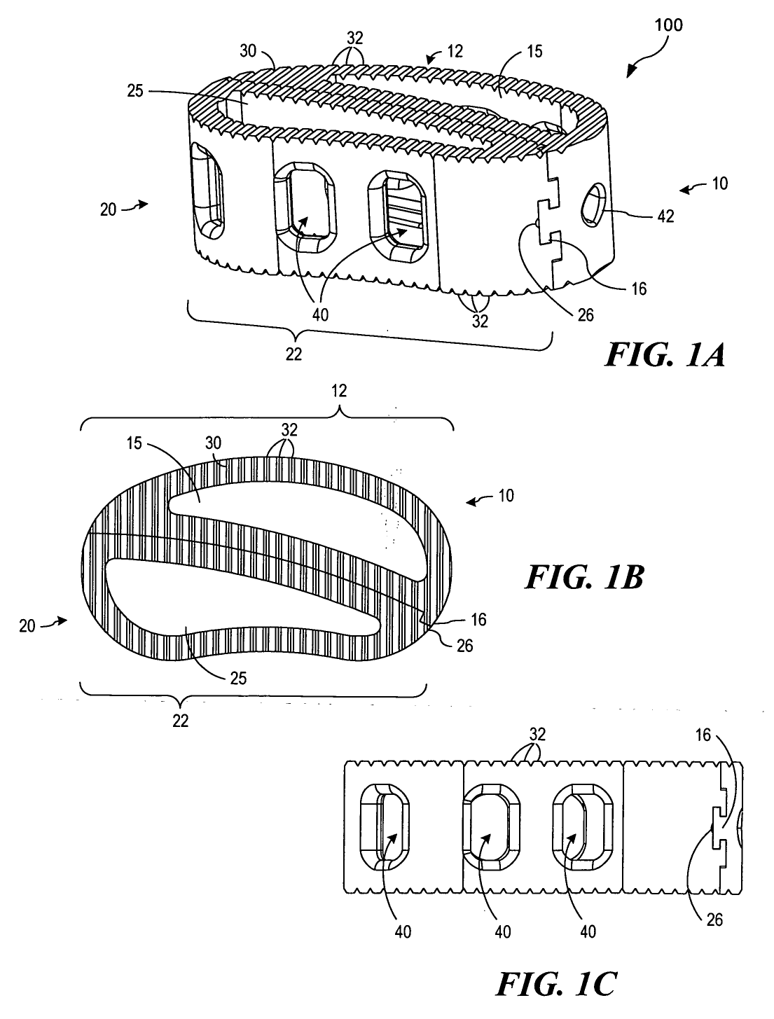

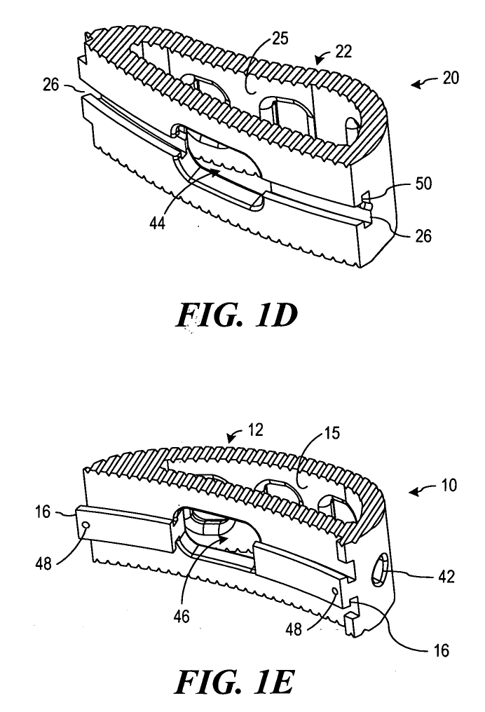

FIGS. 1A and 1B depict a spinal implant 100. In certain embodiments, the spinal implant 100 may be inserted between adjacent vertebra from a posterior approach. In some procedures, a Transforaminal lumbar interbody fusion (TLIF) surgery may be performed. In a TLIF approach, one entire facet joint may be removed. Removal of the facet joint, allows visualization into the disc space and access to the disc space. Because one entire facet is removed, typically such procedures are only performed on one side of the spine.

In certain procedures, the surgeon may perform a posterior lumbar interbody fusion (PLIF). In such procedures, the spine is approached through an incision in the midline of the back and the left and right lower back muscles (erector spinae) are stripped off the lamina on both sides and at multiple levels.

After the spine is approached, the lamina may be removed (laminectomy) which allows visualization of the nerve roots. The facet joints, which are directly over the nerve roots, may then be undercut (trimmed) to give the nerve roots more room. The nerve roots are then retracted to one side and the disc space is cleaned of the disc material. The spinal implant 100 may then be inserted into the disc space.

As illustrated in FIGS. 1A and 1B, there is a first part 10 and second part 20 of an interconnecting multi-part spinal implant 100. FIG. 1A depicts an isometric view of the multi-part spinal implant 100, and FIG. 1B depicts a top view of the multi-part spinal implant 100. First part 10 has a back portion 12, which can be, but need not be, convexly arcuate to better conform to the shape of the inter-vertebral space into which it is to be inserted. Second part 20 of the interconnecting multi-part spinal implant 100 has a back portion 22, which can have, but need not have, a concave arcuate portion between two convex arcuate portions to better conform to the shape of the inter-vertebral space into which it is to be inserted. As shown in FIG. 1B, the first part 10 and the second part 20 interconnect to form an arcuate connection.

The upper end and lower end of first part 10, and the upper end and lower end of second part 20, can advantageously have a surface 30 having serrations 32 or another relief pattern disposed thereon, to facilitate retaining the first part 10 and second part 20 between the vertebrae (not shown) without unintended slippage. The first part 10 may have a male dove-tail retention 16 on an interconnecting side, and the second part 20 may have a female dove-tail retention slot 26. The female dove-tail retention slot 26 may be sized sized to fit over the male dove-tail retention rail 16, so that it is longitudinally slidably retained thereon.

First part 10 and second part 20 may be generally hollow, having a cavity 15 in first part 10 and a cavity 25 in second part 20, each of which cavities may be open at their upper and lower ends. If desired, cavities 15 and 25 can advantageously be filled with a material conducive to fusion in a manner adhering first part 10 and second part 20 to the adjacent vertebrae (not shown), such as bone slurry, bone morphogenetic protein (BMP) or the like. In certain embodiments, apertures 40 along the back portion 22 of the second part 20 may allow the healing material to flow into or out of the cavity 25. Similar apertures (not shown) on the back portion 12 of the first part 10 may allow the healing material to flow into the cavity 15. In certain embodiments, apertures 40 permit filler material injected into the spinal implant 100 to flow out of the cavities 15 and 25 and into contact with surrounding vertebrae and exterior surfaces of the cage 100. Additional ports, such as port 42, may also allow the healing material to flow into the cavity 15 after insertion.

FIG. 1C depicts a side view of the back portion 22 of the second part 20 of the multi-part spinal implant 100. In certain embodiments, the serrations 32 may reside on the top and bottom sides of the multi-part spinal implant 100. In some embodiments, the apertures 40 provide access into the cavity 25.

FIG. 1D depicts an isometric view of the second part 20 of the multi-part spinal implant 100. FIG. 1E depicts an isometric view of the first part 10 of the multi-part spinal implant 100. As previously described, second part 20 has a female dove-tail retention slot 26, that is sized to fit over male dove-tail retention rail 16 of the first part 10, so that it is longitudinally retained thereon. In some embodiments, the retention rail 16 has at least one protrusion 48 on either end thereof and that mate with depressions 50 formed in either end of the retention slot 26. The protrusions 48 fit into the depressions 50 when the first part 10 and the second part 20 are fully mated so that the two parts of the spinal implant 100 snap together and stay in the desired position. These bumps 48 are an example of a retention method. An alternative embodiment has straight mating surfaces and ratcheting teeth for retention. It should be noted that, although a flat-sided dove-tail shaped retention rail 16 is depicted, the retention rail 16 and the female retention slot 26 could also have curved sides provided that the rail 16 can still be longitudinally slidably retained in the slot 26. Retention rail 16 and retention slot 26 may have any configuration of interlocking shapes that still permit longitudinal sliding. Note that there may be two or more such rails 16 and that the one or more rails and slot 26 may be segmented into two or more mating lengths shorter then the entire length of the parts. Second part 20 may have an aperture 44 and first part 10 may have an aperture 46 that interconnect the cavity 25 and the cavity 15. When the spinal implant 100 is fully interconnected, apertures 44 and 46 match up to provide the interconnection between the two cavities 25 and 15.

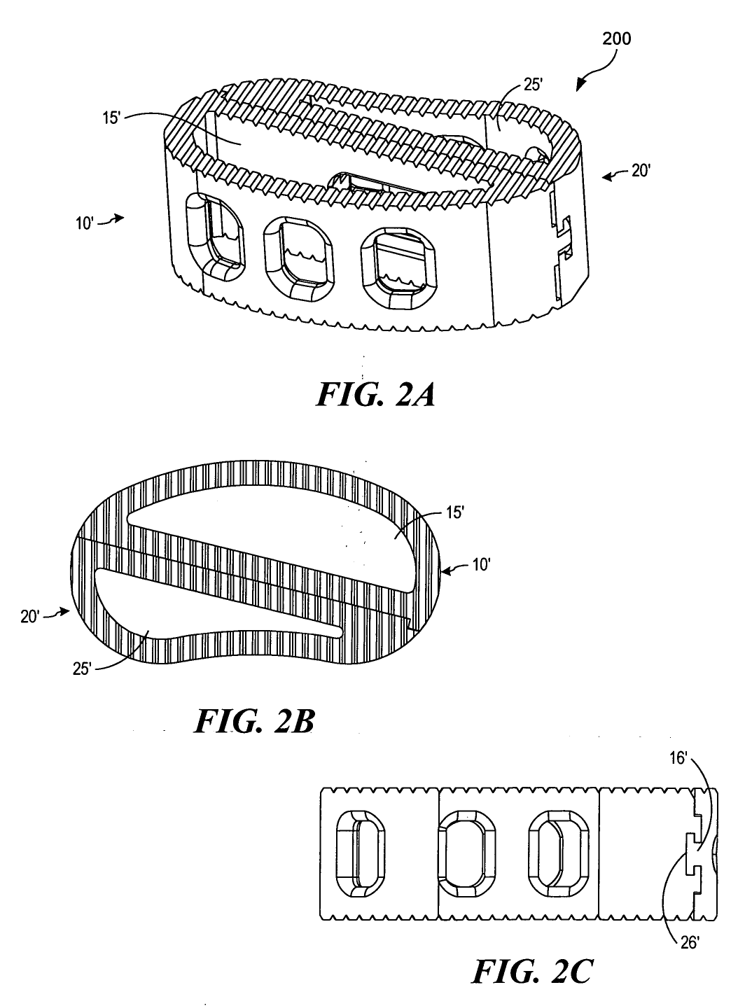

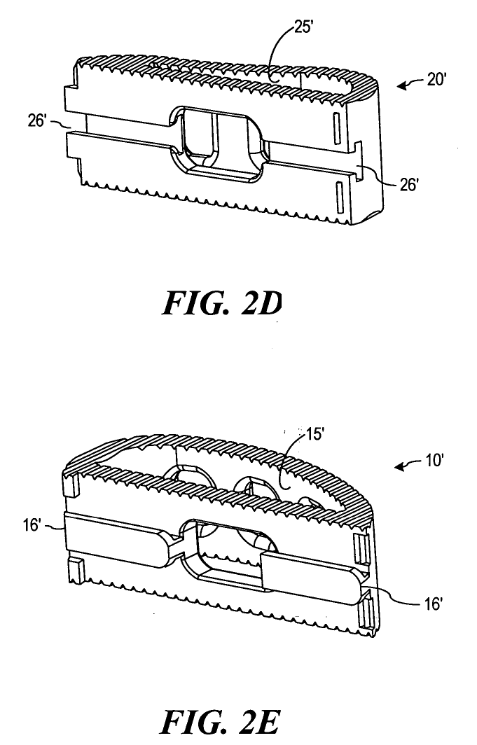

FIGS. 2A-2E depict another embodiment of a spinal implant or LIF cage 200, having components substantially similar to those discussed in connection with and depicted in FIGS. 1A-1E. Such substantially similar components are identified by the same reference numeral, accompanied by a prime (′) designation in FIGS. 2A-2E. FIGS. 2A and 2B depict a first part 10′ and second part 20′ of an interconnecting multi-part LIF cage 200. FIG. 2A depicts an isometric view of the multi-part LIF cage 200, and FIG. 2B depicts a top view of the multi-part LIF cage 200. In certain embodiments, the first part 10′ contains cavity 15′ and second part 20′ contains cavity 25′. FIG. 2C depicts a side view of the back portion 22′ of the second part 20′ of the multi-part LIF cage 200. FIG. 2D depicts an isometric view of the second part 20′ of the multi-part LIF cage 200. The second part 20′ comprises a female retention slot 26′. FIG. 2E depicts an isometric view of the first part 10′ of the multi-part LIF cage 200. The first part 10′ comprises a male dove-tail retention rail 16′. As illustrated in FIG. 2B, the first part 10′ and the second part 20′ interconnect to form a linear connection, in contrast to the arcuate connection illustrated in FIG. 1 B.

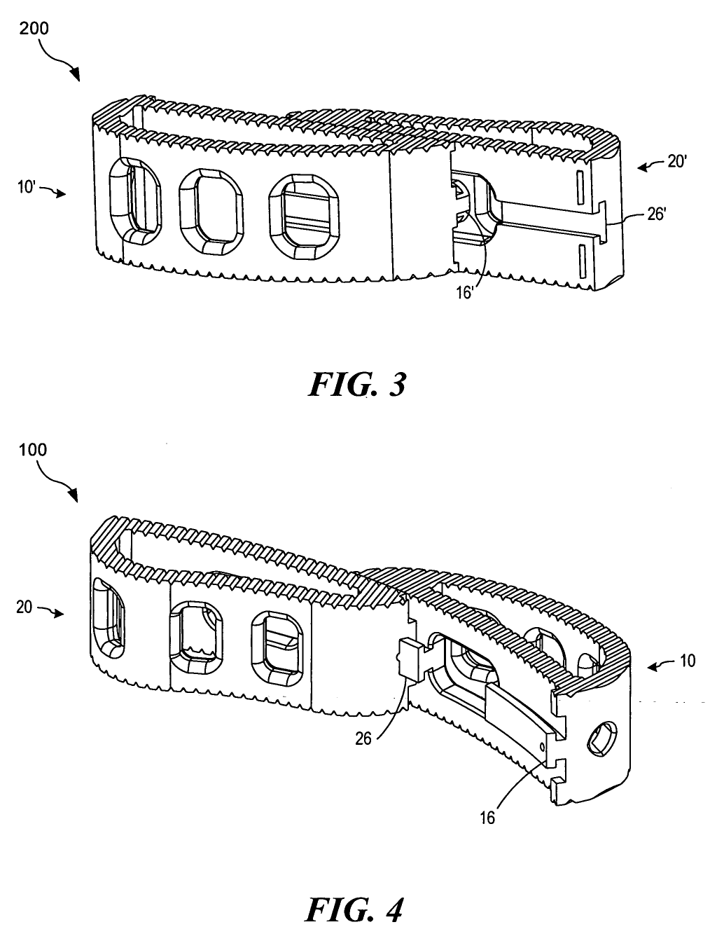

FIG. 3 depicts a first part 10′ and a second part 20′ interconnecting to form a multi-part LIF cage 200. FIG. 3 represents the LIF cage 200 of FIGS. 2A-2E. FIG. 4 depicts a first part 10 and a second part 20 interconnecting to form a multi-part spinal implant 100. FIG. 4 represents the spinal implant 100 of FIGS. 1A-1E.

With reference to FIG. 4, when it is desired to insert spinal implant 100 into a patient, first part 10 and second part 20 are partially interconnected by sliding retention rail 16 of first part 10 part-way into retention slot 26 of second part 20 at their respectively transversely smaller ends. As so connected, the combination of the first part 10 and second part 20 has a smaller maximum transverse thickness than would be the case with both parts fully interconnected. This facilitates surgical insertion of the spinal implant 100 because the smaller maximum transverse thickness requires a smaller surgical access incision.

Once the partially interconnected first part 10 and second part 20 of spinal implant 100 are inserted between the desired vertebrae, the first part 10 and second part 20 must be fully interconnected to reach the fully assembled (snapped together, cojoined, etc.) final configuration, as shown in FIG. 1A. To do so, second part 20 is pushed longitudinally forward while first part 1 is restrained from moving. This causes the slot 26 to longitudinally slide over rail 16 until the respective ends are generally flush, as depicted in FIG. 1A. The position of the fully interconnected spinal implant 100 may then be manually adjusted to ensure that it is in the desired position between the two adjacent vertebrae.

Once the spinal implant 100 is in the desired, final position, a filler material conducive to rapid healing in a manner adhering first part 10 and second part 20 to the adjacent vertebrae (not shown), such as bone slurry, bone morphogenetic protein (BMP) or the like, can be injected into the cavity 15 of first part 10 through port 42 (FIG. 1A). It should be noted that one or both of the first and second parts 10, 20 may be partially or completely filled;with the filler material prior to insertion and placement between the vertebra. Filler material may then be added to fill both parts and, if desired, to cause the filler material to spill out of apertures 40 (FIG. 1A) in the external side walls of the first and second parts 10, 20, to cover all or part of the first and second parts 10, 20, to further enhance stimulation of bone growth.

There are many instruments that can be used to insert these LIF cages 100, 200 into the intervertebral space. Some of these instruments are described in a co-pending and commonly assigned U.S. patent application Ser. No. ______, entitled “INSTRUMENTS FOR INSERTING SPINAL DISC IMPLANTS,” filed ______, the disclosure of which is hereby incorporated herein by reference.

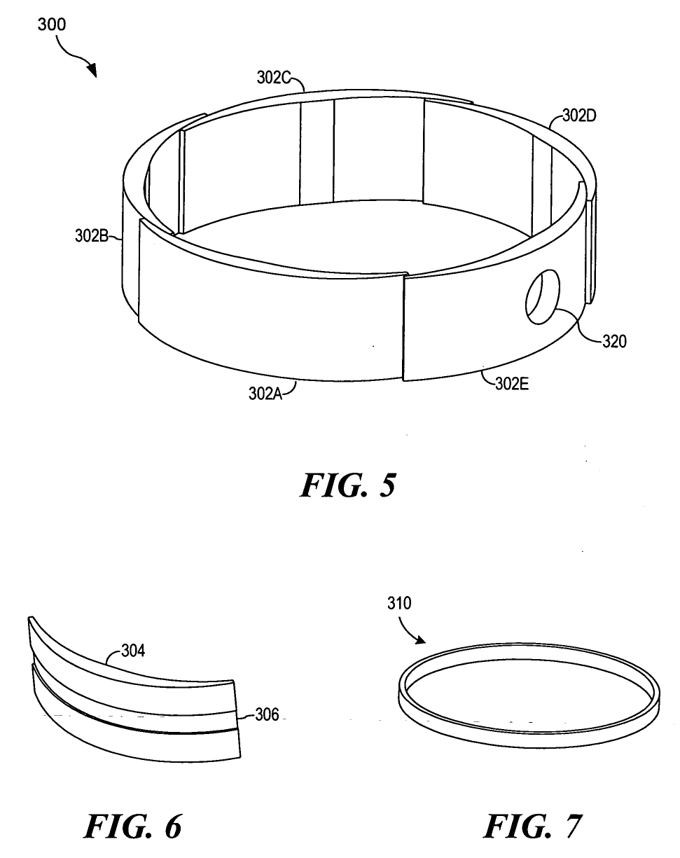

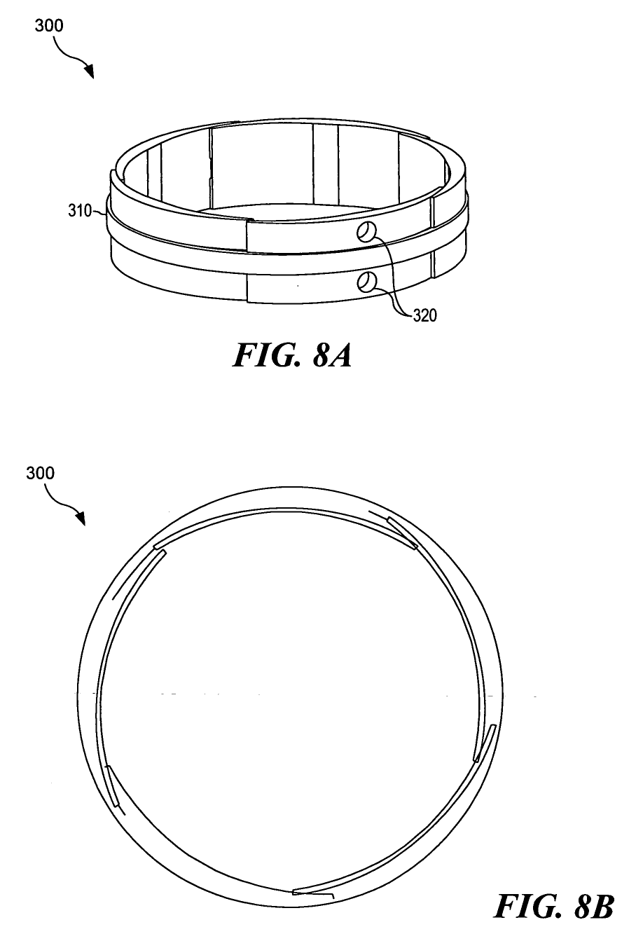

FIG. 5 depicts a perspective view of an alternative embodiment of an expandable cage 300. In this embodiment, the cage 300 has multiple sliding parts 302A-302E. Each of sliding parts 302A-302E is slidably interconnected to its adjacent part by an interconnected slot and rail (not shown). In certain embodiments, a ratchet locking means (not shown) may also be used to interconnect the sliding parts. In FIG. 5, the cage 300 is depicted as assembled to its full-size, final configuration, as it would be installed between the vertebrae. An aperture 320, allows a filler material conducive to rapid healing, such as bone slurry, bone morphogenetic protein (BMP) or the like, to be injected into the cavity of the expandable cage 300. FIG. 6 depicts one sliding part 304 with a groove 306. FIG. 7 depicts a band 310 which may restrain the cage 300. This band 310 is meant to hold the final shape of the embodiment 300. The device would be inserted through the surgical port while collapsed and with the band 310 attached to the outside by some sort of mechanical or adhesive restraint. As the filler or expanding means is applied to attain expansion, the band 310 would act as a restraint to limit the expansion or help the device reach its final desired shape. A circle is shown as the final desired shape for simplicity, however the final or “set configuration” shape could be any closed shape, such as an ellipse. The groove 306 shown for the sliding part 304 may hold a band 310 or other restraining device. In certain embodiments, prior to insertion through a surgical incision, the cage 300 may be collapsed by applying force about the circumference, and then the cage 300 may be retained in the collapsed condition by means of a band 310 or other restraining device (FIG. 7) placed around the circumference of the cage 300. FIG. 8A depicts the band 310 placed around the circumference of the cage 300. When the band or other retraining device is removed, the cage 300 will be allowed to expand to its final configuration, as shown in FIG. 5. FIG. 8B depicts a top view of the expandable cage 300.

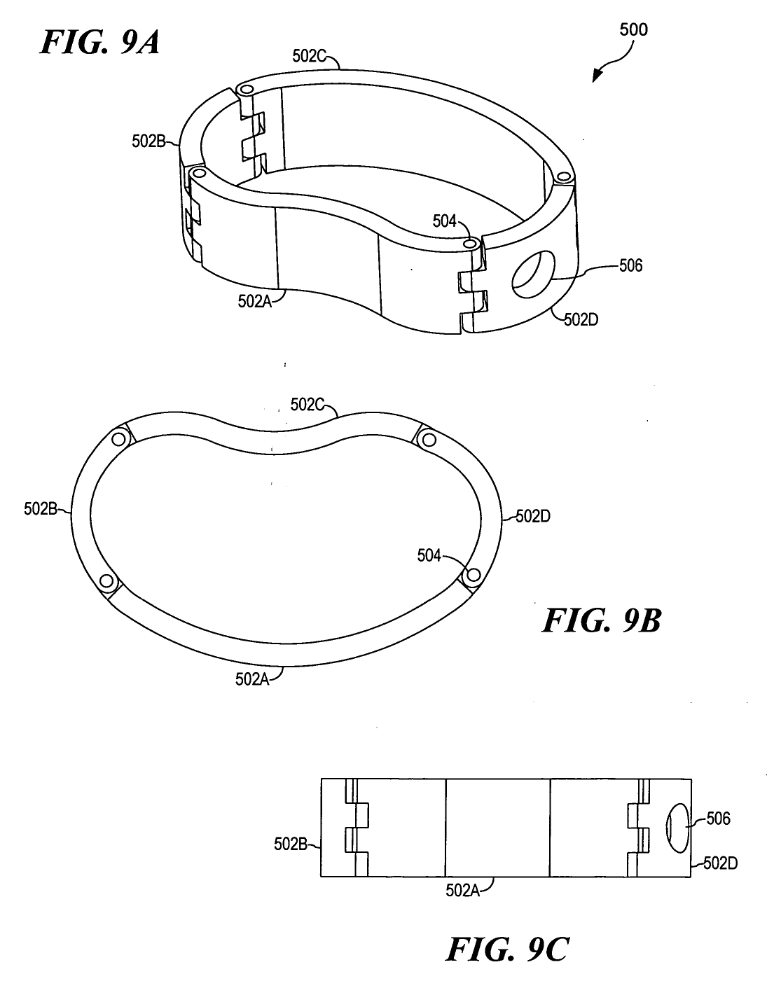

FIG. 9A depicts a perspective view of an alternative embodiment of the expandable cage 500. In this embodiment, the cage 500 has multiple hinged parts 502A-502D. In some embodiments, each of the hinged parts 502A-502D is interconnected to its adjacent part 502 by a pin hinge. A pin hinge attachment is only one embodiment of the present invention. In other embodiments, molded-in hinge pins, double pin-ended links, snap-fit dome-in-socket, and the like can be used to interconnect the hinged parts. Accordingly, a pin 504 holds the hinged parts 502 together, so as to be pivotable with respect to each other. An aperture 506 allows a filler material, such as bone slurry, BMP or the like, to be injected into the cavity of the expandable cage 500. FIG. 9B depicts a top view of the expandable cage 500. FIG. 9C depicts a side view of the expandable cage 500.

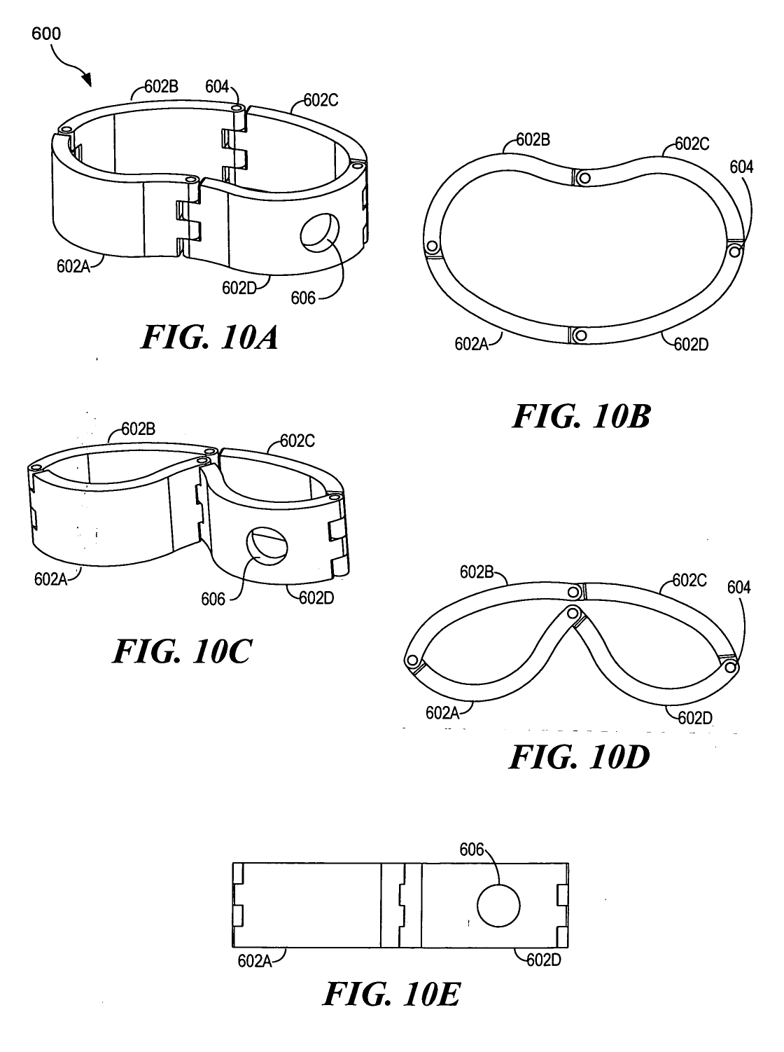

FIG. 10A depicts a perspective view of an alternative embodiment of the expandable cage 600. FIG. 10B depicts a top view of the expandable cage 600. FIGS. 10A-B illustrate the expandable cage 600 in a set or expanded configuration. FIG. 10C depicts a perspective view of the expandable cage 600 in an insertion or a contracted state, and FIG. 10D depicts a top view of the expandable cage 600 in a contracted state. In the contracted state, the expandable cage 600 resembles an hourglass shape and has a greatly reduced cross-sectional width. In certain embodiments, by applying pressure to the cage 600, the cage may be collapsed to the position depicted in FIGS. 10C-D. In this embodiment, the cage 600 has multiple hinged parts 602A-D. Each of the hinged parts 602 is interconnected to its adjacent part 602 by a pin hinge. A pin hinge attachment is only one embodiment of the present invention. In other embodiments, molded-in hinge pins, double pin-ended links, snap-fit dome-in-socket, and the like can be used to interconnect the hinged parts. Accordingly, a pin 604 holds the hinged parts 602 together, so as to be pivotable with respect to each other. An aperture 606 allows a filler material conducive to rapid healing, such as bone slurry, BMP or the like, to be injected into the :cavity of the expandable cage 600. FIG. 10E depicts a side view of the expandable cage 600.

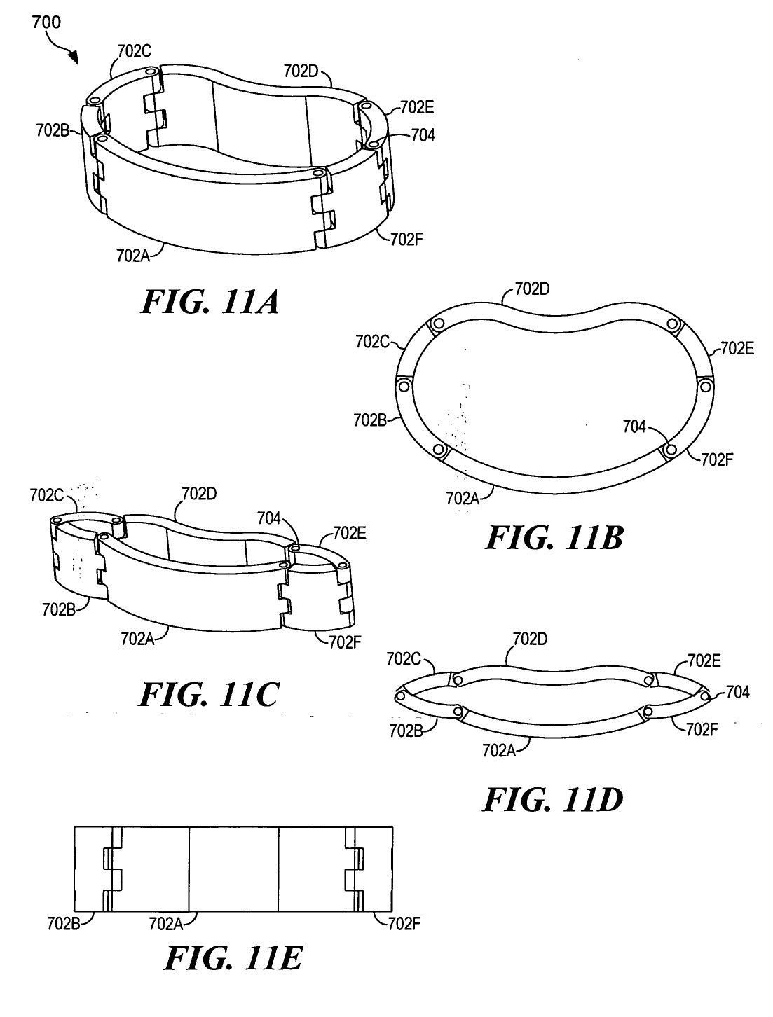

FIG. 11A depicts a perspective view of an alternative embodiment of the expandable cage 700. FIG. 11B depicts a top view of the expandable cage 700. FIGS. 10A-B illustrate the expandable cage 700 in an expanded state. FIG. 11C depicts a perspective view of the expandable cage 700 in a contracted state, and FIG. 11D depicts a top view of the expandable cage 700 in a contracted state. In the contracted state, the expandable cage 700 has a greatly reduce cross-sectional width. Thus by applying pressure to the cage 700, the cage may be collapsed to the position depicted in FIGS. 11C-D. In this embodiment, the cage 700 has multiple hinged parts 702A-702F. In certain embodiments, each of the hinged parts 702A-702F is interconnected to its adjacent part by a pin hinge. A pin hinge attachment is only one embodiment of the present invention. In other embodiments, molded-in hinge pins, double pin-ended links, snap-fit dome-in-socket, and the like can be used to interconnect the hinged parts. Accordingly, a pin 704 holds the hinged parts 702 together, so as to be pivotable with respect to each other. FIG. 1I E depicts a side view of the expandable cage 700.

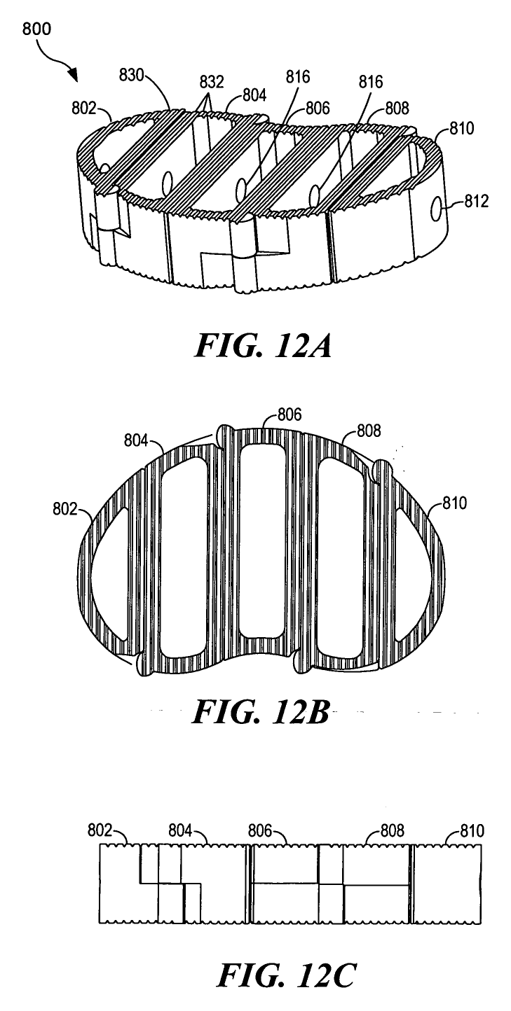

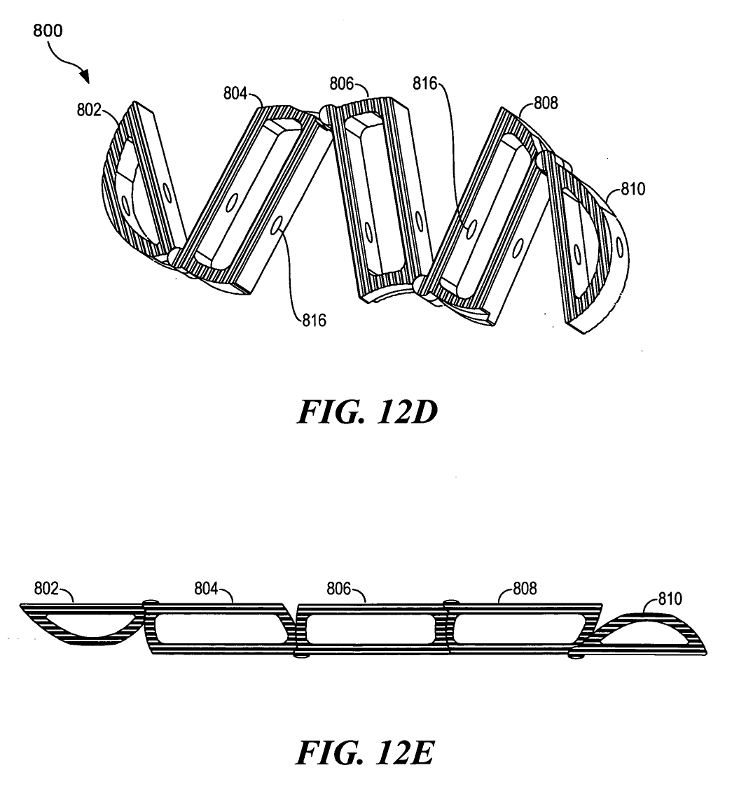

FIG. 12A depicts a perspective view of another embodiment of an accordion-configuration expandable cage 800. FIG. 12B is a top view of the accordion-configuration expandable cage 800. FIG. 12C is a side view of the accordion-configuration expandable cage 800. In certain embodiments, the expandable cage 800 may have multiple hinged parts 802, 804, 806, 808, and 810 which are shown in a foldable configuration. FIGS. 12A-C illustrate the cage 800 in its set or expanded configuration, as it would be installed in the intertebral disc space. In certain embodiments, the hinged parts 802, 804, 806, 808, and 810 may be interconnected by pin hinges. A pin hinge attachment is only one embodiment of the present invention. In other embodiments, molded-in hinge pins, double pin-ended links, snap-fit dome-in-socket, and the like can be used to interconnect the hinged parts. The cage 800 may advantageously have a surface 830 having serrations 832 or another relief pattern disposed thereon, to facilitate retaining the cage 800 between the vertebrae (not shown) without unintended slippage.

FIG. 12D depicts a perspective view of the expandable cage 800, where the cage 800 is partially folded towards its full size or final configuration as it would be installed between the vertebrae. FIG. 12E depicts a plan view of the expandable cage 800, where the multiple hinged parts 802-810 are arranged longitudinally in a line, which is one possible insertion configuration. Alternatively, the parts 802-810 may be arranged in a curve. Accordingly, the cage 800 is extended so as to have a small transverse width, for insertion through a surgical incision. As depicted in FIGS. 12A-E, the hinged parts may each be hollow. As depicted in FIGS. 12A and 12D, part 810 has a port 812 in a side thereof. Once the assembly is finally positioned, a material conducive to rapid healing in a manner adhering hinged parts 802-810 to the adjacent vertebrae (not shown), such as bone slurry, BMP or the like, may be injected through a lumen. This material may be injected prior to or after insertion. From there, cross-connect ports 816 between each of the parts 802-810 permit passage of the material from parts 810 to 808, from 808 to 806, from 806 to 804, and from 804 to 802 until all the cavities of the cage 800 are filled.

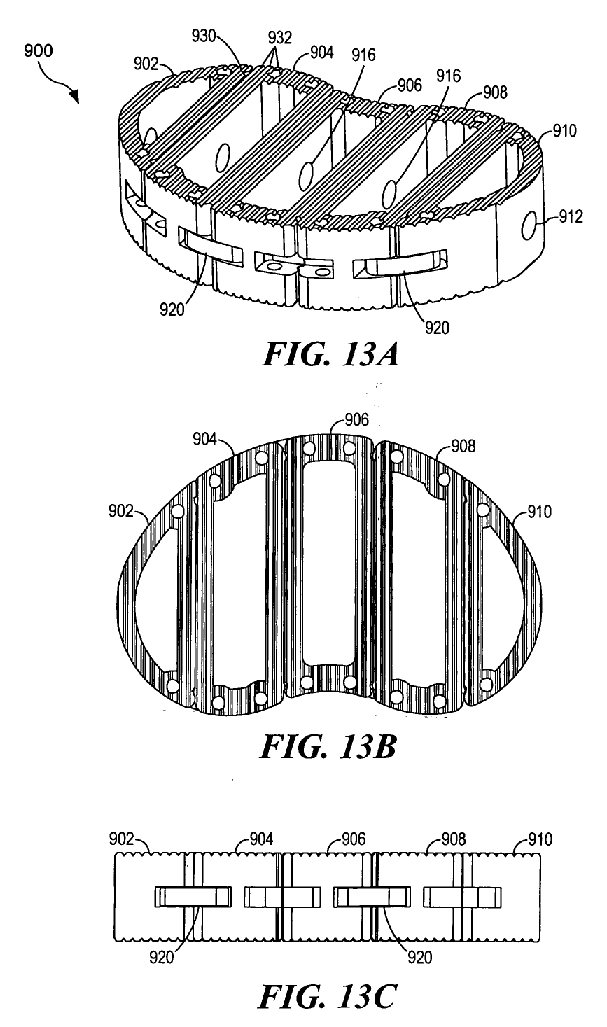

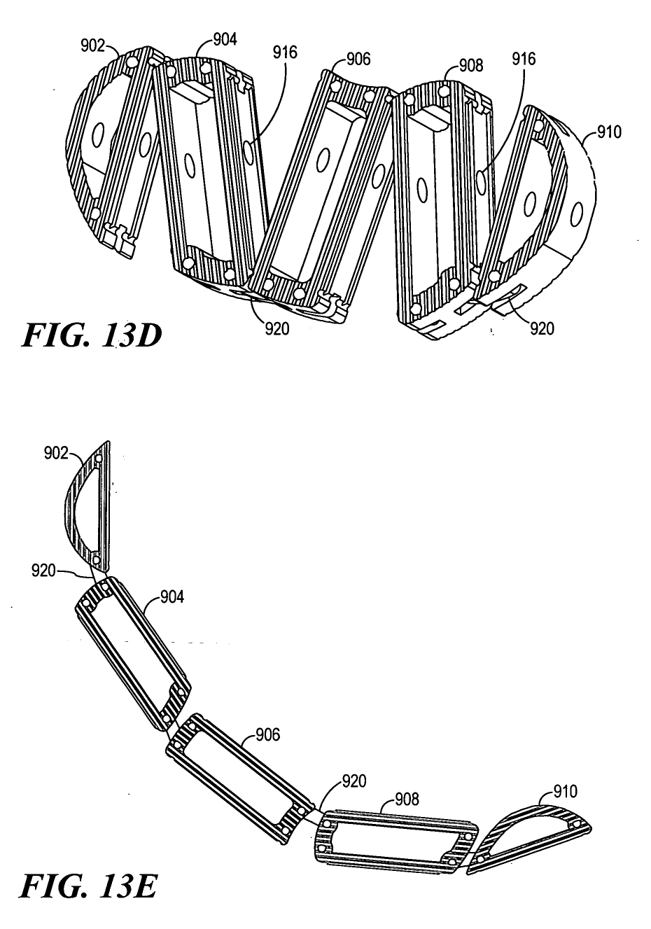

FIG. 13A depicts a perspective view of another embodiment of an accordion-configuration expandable cage 900. FIG. 13B is a top view of the accordion-configuration expandable cage 900. FIG. 13C is a side view of the accordion-configuration expandable cage 900. In certain embodiments, this expandable cage has multiple hinged parts 902, 904, 906, 908, and 910 in a foldable configuration. FIGS. 13A-C illustrate the cage 900 in its set or expanded final configuration, as it would be installed in the vertebrae. The hinged parts 902, 904, 906, 908, and 910 are interconnected by multiple double pin-ended links 920. Accordingly, one double pin-ended link 920 holds part 910 and 908 together. The cage 900 may advantageously have a surface 930 having serrations 932 or another relief pattern disposed thereon, to facilitate retaining the cage 900 between the vertebrae (not shown) without unintended slippage.

FIG. 13D depicts a perspective view of the expandable cage 900, where the cage 900 is partially folded towards its full size or final configuration as it would be installed between the vertebrae. FIG. 13E depicts a plan view of the expandable cage 900, where the multiple hinged parts 902-910 are arranged longitudinally in a line. Accordingly, the cage 900 is extended so as to have a small transverse width, for insertion through a surgical incision. The double pin-ended links 920 interconnect the hinged parts 902-910. As depicted in FIGS. 13A-E, the hinged parts may each be hollow. As depicted in FIGS. 13A and 13D, part 910 has a port 912 in a side thereof. Once the assembly is finally positioned, a material conducive to rapid healing in a manner adhering hinged parts 902-910 to the adjacent vertebrae (not shown), such as bone slurry, BMP or the like, may be injected through a lumen. From there, cross-connect ports 916 between each of the parts 902-910 permit passage of the material from parts 910 to 908, from 908 to 906, from 906 to 904, and from 904 to 902 until all the cavities of the cage 900 are filled.

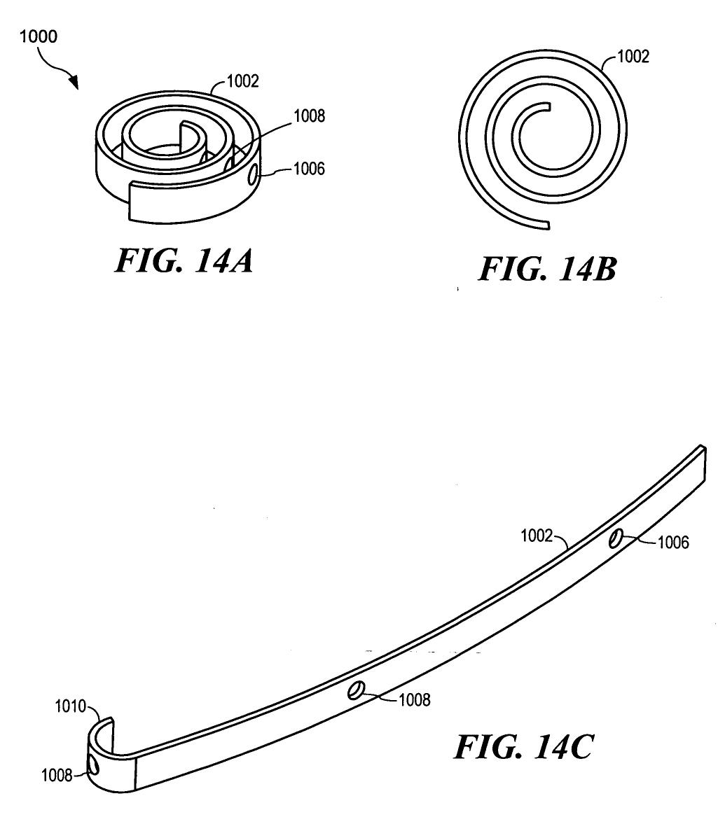

FIG. 14A depicts a perspective view of an alternative embodiment of an expandable cage 1000. FIG. 14B depicts a plan view of the expandable cage 1000. FIGS. 14A-B illustrate the cage 1000 in its fully expanded final configuration, as it would be installed in the vertebrae. In certain embodiments, the cage 1000 comprises at least one rectangular piece of material 1002 that may be flexible enough to bend into a set or spiral configuration upon an actuating event. For instance, the cage 1000 may be formed of using a memory metal, such as Nitinol . FIG. 14C depicts a perspective view of the expandable cage 1000, where the cage is arranged longitudinally in a line. An additional half-circle shaped piece 1010 is connected to the rectangular piece 1002. Accordingly, the cage 1000 is extended so as to have a small transverse width, for insertion through a surgical incision. As the cage enters the intervertebral space, the rectangular piece 1002 may bend and curl to form the spiral configuration in FIG. 14A. As depicted in FIGS. 14A and 14C, the rectangular piece has a port 1006. Once the assembly is finally positioned, a material, such as bone slurry, BMP or the like, can be injected through a lumen. From there, cross-connect ports 1008 inside of the cage 1000 permit passage of the material from one cavity to the next cavity. Ultimately, all of the cavities of the cage 1000 may be filled.

There are many instruments that can be used to insert these expandable cages 300, 500, 600, 700, 800, 900 and 1000 into the intervertebral space. Some of these instruments are described in a co-pending and commonly assigned U.S. patent application Ser. No. ______, entitled “INSTRUMENTS FOR INSERTING SPINAL DISC IMPLANTS,” filed ______, the disclosure of which is hereby incorporated herein by reference.

It is important to note that any such advantages and benefits described in this application may not apply to all embodiments of the invention. When the word “means” is recited in a claim element, Applicant intends for the claim element to fall under 35 U.S.C. § 112, paragraph six. Often a label of one or more words precedes the word “means.” The word or words preceding the word “means” is a label intended to ease referencing of claim elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word means are not intended to fall under 35 U.S.C. § 112, paragraph 6.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

What is claimed is:

1. An expandable spinal implant comprising:
a first part;
a second part; and
a means for movably interconnecting the first part and the second part, wherein the implant has a smaller transverse thickness when the first part and the second part are partially interconnected than when the first part and the second part are fully interconnected.

2. The expandable spinal implant of claim 1, wherein the first and second parts each comprise at least one cavity.

3. The expandable spinal implant of claim 2, wherein the first and second parts each comprise at least one first aperture, wherein the at least one first aperture enables material to flow into and out of the at least one cavity.

4. The expandable spinal implant of claim 3, wherein the first and second parts each comprise at least one second aperture, wherein the at least one second aperture enables material to flow back and forth between the cavity of the first part and the cavity of the second part.

5. The expandable spinal implant of claim 1, wherein the means for movably interconnecting the first part and the second part further comprises at least one portion of a male retention rail and at least one portion of a female retention slot.

6. An expandable spinal implant comprising:
a plurality of curved moveable parts;
means for interconnecting the curved moveable parts, wherein each curved moveable part is interconnected to two adjacent curved sliding parts to create a substantially oval shape;
wherein by applying a pressure to at least one surface of one of the curved moveable parts, the curved moveable parts slide with respect to each other to increase or decrease the circumference of the spinal implant.

7. The expandable spinal implant of claim 6, wherein the means for interconnecting the curved moveable parts comprises a plurality of interconnecting slots and rails.

8. The expandable spinal implant of claim 6, wherein the means for interconnecting the curved moveable parts further comprises a means for ratchet locking each curved moveable part to an adjacent curved moveable part.

9. The expandable spinal implant of claim 6, wherein the expandable spinal implant further comprises at least one aperture.

10. An expandable spinal implant comprising:
a plurality of curved hinged parts;
means for interconnecting the curved hinged parts, wherein each curved hinged part is interconnected to two adjacent curved hinged parts to create a substantially oval shape;
wherein by applying a pressure to at least one surface of a curved hinged part, the plurality of curved hinged parts collapse towards the center of the spinal implant or the plurality of curved hinged parts expand away from the center of the implant.

11. The expandable spinal implant of claim 10, wherein the means for interconnecting the curved hinged parts further comprises a plurality of pin hinges.

12. The expandable spinal implant of claim 10, wherein the means for interconnecting the curved hinged parts further comprises a plurality of double pin-ended links.

13. The expandable spinal implant of claim 10, wherein the expandable spinal implant further comprises at least one aperture.

14. An expandable spinal implant comprising:
a plurality of hinged parts comprising a length and a width, wherein each of the hinged parts comprises a length that is larger than a width;
means for interconnecting the hinged parts,
wherein when the expandable spinal implant is expanded the plurality of hinged parts are assembled such that a length of each hinged part is substantially adjacent to a length of an adjacent hinged part; and
wherein when the expandable spinal implant is contracted the plurality of hinged parts are assembled such that a width of each hinged part is substantially adjacent to a width of an adjacent hinged part.

15. The expandable spinal implant of claim 14, wherein at least one hinged part of the plurality of hinged parts comprises at least one cavity.

16. The expandable spinal implant of claim 15, wherein at least one hinged part of the plurality of hinged parts comprises at least one aperture.

17. The expandable spinal implant of claim 14, wherein the means for interconnecting the hinged parts further comprises a plurality of pin hinges.

18. The expandable spinal implant of claim 14, wherein the means for interconnecting the hinged parts further comprises a plurality of double pin-ended links.

19. An expandable spinal implant comprising:
at least one rectangular piece of material with a smaller transverse width than a length, wherein the rectangular piece of material bends into a spiral configuration when a force is applied to at least a portion of the rectangular piece of material.

20. The expandable spinal implant of claim 19, wherein the at least one rectangular piece of material comprises at least one aperture.
